(19) United States
(12) Patent Application Publication
(10) Pub. No.: US 2022/0384043 A1
(43) Pub. Date: Dec. 1, 2022

(54) SYSTEMS AND METHODS FOR ENHANCED PHOTODETECTION SPECTROSCOPY USING DATA FUSION AND MACHINE LEARNING

(71) Applicant: Lightsense Technology, Inc., Tucson, AZ (US)

(72) Inventors: Wade Martin Poteet, Vail, AZ (US); Terje A. Skotheim, Tucson, AZ (US)

(21) Appl. No.: 17/825,983

(22) Filed: May 26, 2022

Related U.S. Application Data
(60) Provisional application No. 63/194,714, filed on May 28, 2021.

(51) Int. Cl. G16H 50/20

(52) U.S. Cl. CPC G16H 50/20

(57) ABSTRACT

Embodiments of this invention relate generally to a method for detection of pathogens, biomarkers, or any compound using data fusion and machine learning. The method includes generating, with a first miniature UV absorption spectrometer of a multi-spectral optical device, a first absorption spectral output based on receiving an absorbance light channel from a sample, generating, with a second miniature UV fluorescence spectrometer of the multi-spectral optical device, a second emission spectral output based on receiving an emission light channel from the sample and performing, with the multi-spectral optical device, data fusion between the first absorption spectral output and the second emission spectral output to generate fused data.

### RELATED APPLICATIONS

This application claims the priority of U.S. Provisional Application No. 63/194,714, filed May 28, 2021, the contents of which are incorporated by reference herein.

This invention was made with government support under contract SP4701-21-P-0029 awarded by Defense Logistics Agency. The government has certain rights in the invention.

### FIELD OF THE INVENTION

Embodiments of this invention relate generally to an enhanced photodetection spectroscopy for detection of pathogens, biomarkers, or any compound using data fusion and machine learning.

### BACKGROUND

Ultraviolet fluorescence refers to the process where a substance is exposed to sufficient energy at ultraviolet and visible wavelengths between 200 nm and 900 nm and this interaction with the substance results in absorption of that energy and subsequent emission from that substance at a longer wavelength than the applied wavelength. Ultraviolet specular reflection refers to the process wherein certain wavelengths of ultraviolet energy are reflected and others either partially or totally absorbed. Other analytical methods involve absorption of certain wavelengths and not other wavelengths as a substance is illuminated with ultraviolet energy, and this technique is generally employed as an analytical chemistry tool to determine the presence of a particular substance in a sample and, in many cases, to quantify the amount of the substance present. Ultraviolet-visible spectroscopy is particularly common in analytical applications. There are a wide range of experimental approaches for measuring absorption spectra. The most common arrangement is to direct a generated beam of radiation at a sample and detect the intensity of the radiation that passes through it. The transmitted energy can be used to calculate the wavelength-dependent absorption. Raman scattering spectroscopy is also used for substance identification, and excels at identifying individual substances, but significant data processing is required to separate substances in a complex mixture, and the technique is expensive.

Standard spectrometer techniques have difficulty when the target substance is present at a low concentration within a mixture of a large number of distractors, such as a virus in a biological fluid like saliva.

### SUMMARY

Embodiments of this invention relate generally to methods of an enhanced photodetection spectroscopy for detection of pathogens, biomarkers, or any compound using data fusion and machine learning. In one example, a method utilizes data fusion and machine learning for identifying and measuring a virus load of a sample. The method includes generating, with a first miniature UV absorption spectrometer of a multi-spectral optical device, a first absorption spectral output based on receiving an absorbance light channel from a sample, generating, with a second miniature UV fluorescence spectrometer of the multi-spectral optical device, a second emission spectral output based on receiving an emission light channel from the sample and performing, with the multi-spectral optical device, data fusion between the first absorption spectral output and the second emission spectral output to generate fused data.

Other features and advantages of embodiments of the present invention will be apparent from the accompanying drawings and from the detailed description that follows below. Other features and advantages of embodiments of the present invention will be apparent from the accompanying drawings and from the detailed description that follows below.

### BRIEF DESCRIPTION OF THE DRAWINGS

The accompanying drawings are included to provide further understanding of the invention and constitute a part of the specification. The drawings listed below illustrate embodiments of the invention and, together with the description, serve to explain the principles of the invention, as disclosed by the claims and their equivalents.

FIG. 1 illustrates a block diagram of an enhanced photodetection spectrometer (EPS) system in accordance with one embodiment.

FIG. 2 illustrates Spectrometer building blocks for multi-spectral architecture (EPS) in accordance with one embodiment.

FIG. 3 illustrates components of UVF/UVA EPS system 300 for viral detection that can be used to detect SARS-CoCV-2 coronavirus in saliva in accordance with one embodiment.

FIG. 4 illustrates components of a compact EPS detector system 400 in accordance with one embodiment.

FIG. 5 illustrates a diagrammatic representation of a machine in the exemplary form of a computer system or device 600 within which a set of instructions, for causing the machine to perform any one or more of the methodologies discussed herein, may be executed, in accordance with one embodiment.

FIG. 6A illustrates plots of the absorbance spectra for the various viruses in saliva solutions, with a 1:5 ratio in accordance with one embodiment.

FIG. 6B illustrates that the amplitude (and less so the shape) of the spectra can change in absorbance significantly with respect to the ratio, with absorbance decreasing as the virus becomes more diluted in accordance with one embodiment.

FIGS. 7A-7F illustrate fluorescence (emission-excitation) spectra for the 6 viruses (including CoV-2), where X and Y axes represent the excitation and emission wavelengths, respectively, and the Z axis is the intensity in accordance with one embodiment.

FIG. 8 illustrates a process for taking spectra from each type of virus, and simulating variation in the spectra due to different types of multiplicative and additive noise.

FIGS. 9A, 9B, and 9C show the results of a PCA feature extraction in terms of scatter plots visualizing the principal component analysis.

FIG. 10 illustrates how Convolutional Neural Network (CNN), Long Short Term Memory Network (LSTM), and Gated Recurrent Unit (GRU) layers are optimized to take input spectra and output the same spectra, but after going through a compression/bottleneck stage in the middle of the neural network.

FIG. 11 illustrates a machine learning pipeline in accordance with one embodiment.

FIG. 12 illustrates a method for operations of a handheld multi-spectral optical device in accordance with one embodiment.

### DETAILED DESCRIPTION

Testing for viral pathogens (e.g., Coronavirus) is slow and expensive causing costly shutdowns. An absence of rapid testing for bacterial pathogens (e.g., E.coli, Listeria, Salmonella) endangers our food supply. Also, the field detection technology for illicit drugs is inadequate, endangering lives.

The present design relates generally to the field of chemical detection, inspection, and classification. The present design provides detection of pathogens (e.g., coronavirus, bacterial pathogens such as E. coli, salmonella, listeria, etc.) in a sample (e.g., biological sample, saliva) with high accuracy and sensitivity with an optical instrument. Clinical staff is not needed for operation of this optical instrument. The measurement will take no more than 1-2 minutes from beginning to end and cost very little per measurement. A low cost disposable for a sample is part of the detection system. A radical new spectroscopy architecture integrates 2 or more (miniaturized) spectrometer optical components into one instrument, performs multimodal data fusion on the 2 or more different types of spectra and uses machine learning for pattern recognition and identification.

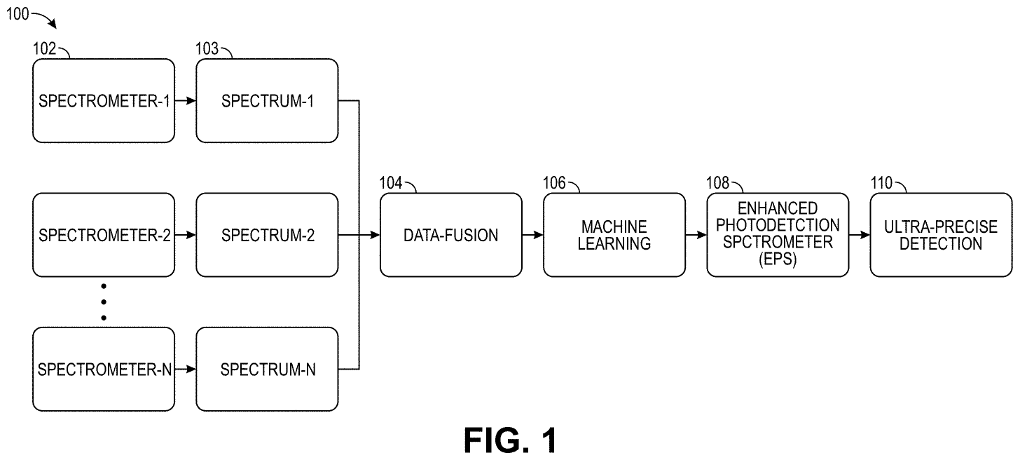

FIG. 1 illustrates a block diagram of an enhanced photodetection spectrometer (EPS) system in accordance with one embodiment. The enhanced photodetection spectrometer 100 includes multiple spectrometers 102 (e.g., spectrometer-1, spectrometer-2, . . . spectrometer-N) that each generate one of the spectrum output 103 (e.g., spectrum 1, spectrum 2, . . . spectrum N), a data fusion component 104, machine learning 106, enhanced spectrometer 108, and ultra-precise detection 110. A spectrum output from 2 or more of the spectrometers are subjected to data fusion component 104 and AI/machine learning 106 for pattern recognition and data treatment. The output from machine learning can be stored in a cloud database. Predictive models and subscription services will be provided.

The present design demonstrations a radical and pathbreaking new spectroscopy architecture that will lead to a point-of-need (PON) handheld instrument for optical detection of pathogens. In one example, this instrument will use saliva samples on a specially designed, low-cost disposable slide for detection of the presence or absence of coronavirus in 2 minutes or less, eliminating the need for device cleaning. Recent research indicates that the concentration of coronavirus in saliva is at least as high as in nasopharyngeal swabs. Measuring on saliva also provides higher safety for personnel, is less invasive, more rapid, and at least as accurate as chemical-based tests.

The new spectrometer architecture includes a combination of at least two spectral processes, fully integrated, with multimodal data fusion and embedded artificial (AI), integrated into one handheld unit. The spectrometer system is able to identify and quantify the measurement of the targeted substance with high sensitivity and accuracy against a complex background. This will result in both determination of the specific target of interest as well as its quantity in the presence of other substances down to very low levels of concentration that would not be possible with a single spectroscopy. This is based on a multispectral architecture, termed Enhanced Photoemission Spectroscopy (EPS) and is illustrated in FIG. 1. The EPS results in sensitivity increase by approximately 100,000 compared to a single spectroscopy.

The key elements of the innovation are:

a radical new multispectral architecture that provides unique capabilities for identifying and quantifying substances, in particular viral pathogens in complex biological fluids;

path-breaking UV photoemission & reflection spectrometer platform;

innovative miniature UV absorption spectrometer system that utilizes a common light source with the UV photoemission spectrometer;

novel AI-based integrated analysis algorithms for multimodal data fusion and rapid analysis of substances, including viruses, down to low concentrations in complex mixtures; and

ability to “learn” the signatures of new viral pathogens not yet in the initial database.

Data fusion is the process of integrating multiple data sources to produce more consistent, accurate, and useful information than that provided by any individual data source. Data fusion processes are often categorized as low, intermediate, or high, depending on the processing stage at which fusion takes place. Data fusion occur when an algorithm uses data from two (or more) different sources, and determines an output based on that data. The most common type of fusion is using information or features from both data sources, and then inputting to the algorithm both features simultaneously at the same time to make a decision. In one exemplary spectral case of data fusion, one spectra has peaks in one region, and another spectra has peaks in a different region, and your decision needs to know not only that there are peaks in these two regions (that's a 1+1=2 case or analyzing the data independent of each other and combining the results), but how these two spectra are jointly correlated with one another. Principal component features from one spectra can be combined with the principal component features of another spectra, and then observe how these features are jointly clustered in feature space (i.e. how the combined features helped improve discriminative clusters for different viruses). A data analysis algorithm can determine which features to extract from each spectra, and these features will be different if you determine these features by analyzing both spectra simultaneously versus analyzing each spectra one at a time.

The present design provides a unique and proprietary advanced micro-electromechanical system (MEMS) technology having the capability to design and produce high performance handheld (pocket-size) UV and Mid-IR spectrometers for a fraction of the cost of equivalent benchtop and handheld standard instruments. A MEMS is a miniature machine that has both mechanical and electronic components. Physical dimensions of a MEMS can range from several millimeters to less than one micrometer.

The miniaturized spectrometer platforms form the key building block modules for design of the radical new integrated multispectral architecture that is the subject of this patent application. The following provides a brief description of each module.

UV Photoemission-Reflection Spectrometer:

The UV Photoemission-Reflection spectrometer platform incorporates two spectroscopies: narrowband UV fluorescence excitation & detection using custom-made narrow-bandpass filters; and UV reflection. This patented design is described further in U.S. application Ser. No. 16/921,614, which is incorporated by reference herein. The UV Photoemission-Reflection spectrometer platform is highly effective in eliminating the background clutter and noise that is typical for standard broadband UV fluorescence. This platform forms the basis for a recently launched handheld, “point-and-shoot” detector of methamphetamine designed for Law Enforcement. The UV Photoemission-Reflection spectrometer platform is the size of a smartphone and is ruggedized for field use. The integration of two spectroscopies, UV photoemission and reflection, results in performance far beyond that of competing handheld Raman spectrometers such as TruNarc from Thermo Fisher, at a significantly lower price. The optical instrument of the present design can include UV Absorption Spectrometer and UV absorption will add a significant data stream to the multimodal spectral integration.

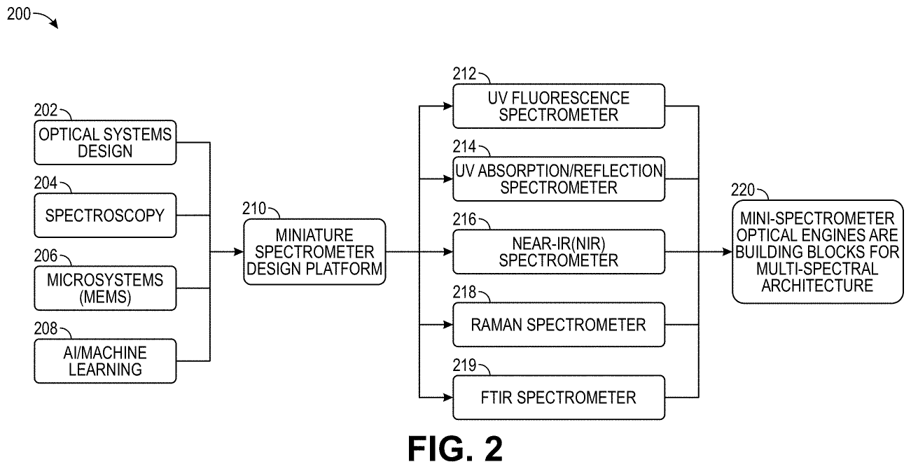

FIG. 2 illustrates Spectrometer building blocks for multi-spectral architecture (EPS) 200 in accordance with one embodiment. The spectrometer building blocks include optical systems design 202, spectroscopy 204, microsystems (MES) 206, and AI/machine learning 208. A miniature spectrometer design platform 210 utilizes multiple spectrometers including UV Fluorescence spectrometer 212, UV absorption/reflection spectrometer 214, a near-IR (NIR) spectrometer 216, a Raman spectrometer 218, or Fourier transform infrared (FTIR) spectrometer 219.

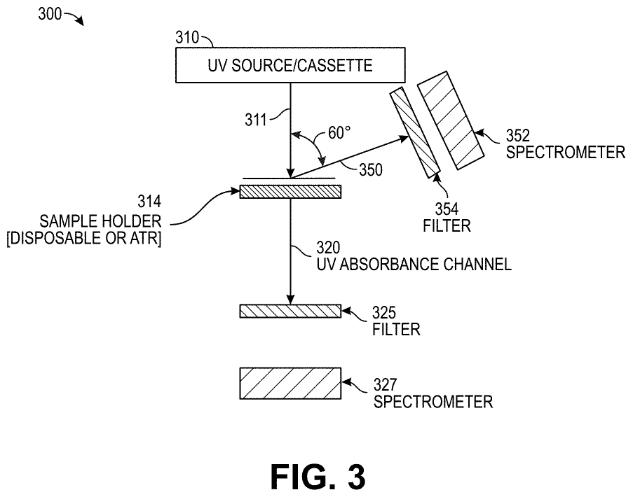

FIG. 3 illustrates components of UVF/UVA EPS system 300 for viral detection that can be used to detect SARS-CoCV-2 coronavirus in saliva in accordance with one embodiment. The system 300 includes a UV source/cassette 310, a sample holder 314 (e.g., disposable holder, Si ATR plate) to support or hold a sample, a UV absorbance channel 320, and a UV fluorescent emission channel 350. The channel 320 passes through a linear UV filter 325 to spectrometer 327 having a linear UV detector. The linear UV filter 325 can be separate or integrated with the spectrometer 327. The channel 350 passes through a linear variable UV filter 354 to a spectrometer 352 having a linear UV detector. The linear UV filter 354 can be separate or integrated with the spectrometer 352. In one example, two fluorescence channels were used with two independent excitation wavelengths.

The UV source 310 generates UV light 311 that is directed on the sample of the sample holder 314 and then the light is reflected as the UV fluorescent emission channel 350 or transmitted as the UV absorbance channel 320. The UV detector of the spectrometer 352 receives the fluorescent emission channel 350 and the UV detector of the spectrometer 327 receives the UV absorbance channel 320 in order to identify and characterize pathogens, biomarkers, or any compound.

The sample holder can be a silicon (Si) attenuated total reflection plate (ATR). This plate can be an inexpensive disposable onto which the sample material is applied. In one embodiment, a thin ruggedly antireflection coated Si window is installed in the spectrometer, possibly at an angle to mitigate residual reflections, so that the Si ATR plate can be inserted into the spectrometer and spring-loaded onto this window or another fixed surface for consistent measurements. This embodiment allows for sealing the spectrometer optical train and filling with inert gas to reduce water vapor and CO₂ absorption lines in the spectrum.

Micro-machined Si ATR methods have been shown to provide enhancements in sample absorption of a factor of 2 to 4 compared to typical sample absorption schemes. This present design can also utilize a signal-enhanced Si ATR plate that has been shown to provide a signal/noise enhancement of a factor of 10 to 18 compared to a standard diamond ATR that is used commercially in FT-IR bench instruments.

Etched structures with dimensions smaller than the mid-IR wavelengths are required on the sample side of the plate to achieve this enhancement. The enhanced ATR plate can achieve much higher performance than a standard grating instrument in the MIR.

The structure on the sample side of the enhanced Si ATR plates has been shown to be able to separate plasma/serum from whole blood as effective as centrifuging, opening entirely new avenues for quick and low-cost whole blood analysis.

In one example, the Si ATR plate is based on a double-side-polished (100) silicon wafer with v-shaped grooves of f111g facets on their backside. These facets are formed by crystal-oriented anisotropic wet etching within a conventional wafer structuring process (e.g., typical wafer thickness of 500 μm). These facets are used to couple infrared radiation into and out of the plate. In contrast to the application of the commonly used multiple-internal reflection ATR elements, these elements provide single-reflection measurement at the sample side in the collimated beam. Due to the short light path within the ATR, absorption in the silicon is minimized and allows coverage of the entire mid-infrared region with a high optical throughput, including the range of silicon lattice vibrations from 300 to 1500 cm⁻¹.

In addition to typical ATR applications, i.e., the measurement of bulk liquids and soft materials, the application of this ATR plate serves three purposes: 1) enhance the sample spectral absorption, 2) provide an inexpensive disposable that is convenient for sample application, and 3) present a sufficiently rugged surface that will withstand physician handling.

Thus, the present design relates to a system, process, and method for pathogen and biomarker detection, inspection, and classification. In particular, the present design includes a combination of two or more spectral processes, fully integrated, with multimodal data fusion and embedded artificial intelligence (AI), or machine learning, integrated into one miniature or handheld unit. The miniature EPS system or optical device is much smaller than normal and has millimeter dimensions (e.g., all dimensions of 100 mm or less; 100 mm×100 mm×40 mm).

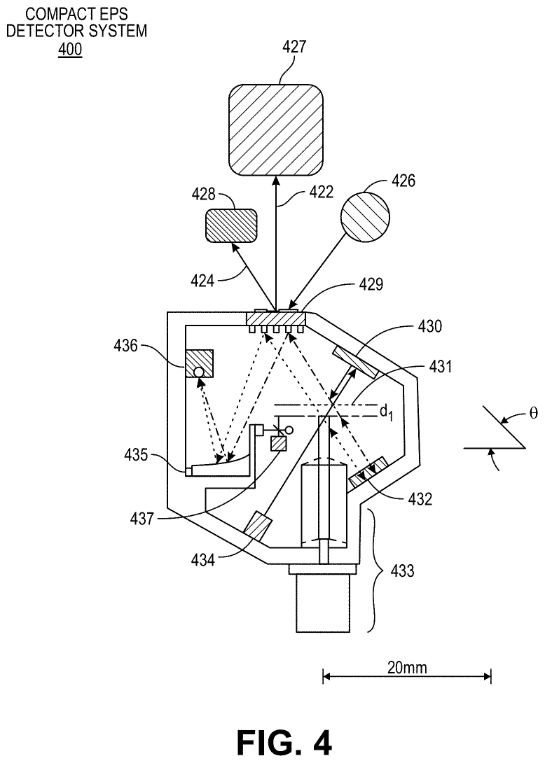

FIG. 4 illustrates components of a compact EPS detector system 400 in accordance with one embodiment. The system 400 includes a UV source 426 (e.g., Xenon UV light source for fluorescence detection system with collimator), a sample holder 429 (e.g., disposable holder, plate, Si ATR plate) having a sample, a UV absorbance channel 422 that is received by an absorbance spectrometer 427 (e.g., UV-Visible Spectrometer) having a detector (or array of detectors), and a UV fluorescent emission channel 424 that is received by a fluorescence spectrometer 428 (e.g., UV Fluorescence spectrometer) having a detector (or array of detectors).

In one example, a disposable sample is positioned on an inexpensive ATR crystal slide. The sample slide potentially contains the pathogen that is inserted into a disposable surround so that the EPS System is contamination-free throughout the measurement process. No sample preparation is required other than applying the patient's fluid onto the disposable inner ATR slide.

The system 400 includes a MEMS IR light source 434 for a FT-IR system, FT-IR fixed mirrors 430 and 432, a movable FT-IR beamsplitter 431 for sample Fourier scan, a beamsplitter Actuator 433 to move the beamsplitter by a distance d1, an Off-Axis Mirror 435 to focus output beam of FT-IR onto spectrometer 436 having an ambient-temperature IR detector, and a Laser Diode alignment Sensor System 437 to provide Laser diode-based alignment for internal interferometer stabilization. The IR light is directed to the beamsplitter 431 and then partially directed back to mirror 432 or partially transmitted through the beamsplitter 431 to the mirror 430. The IR light is then directed from the mirrors 430 and 432, to the beamsplitter at an angle theta to the sample of the sample holder 429.

In this example, three spectrometers each generate spectrum output for 3 spectroscopic processes including FT-IR, UV Fluorescence, and Specular reflection. The miniature spectrometers are coupled to an advanced artificial intelligence data system to reduces false positives and false negatives to a fraction of conventional single-detection process pathogen analysis systems.

In another example, the EPS system could be configured to use only one UV spectrometer in conjunction with the FTIR, either the fluorescence spectrometer or the UV absorption spectrometer.

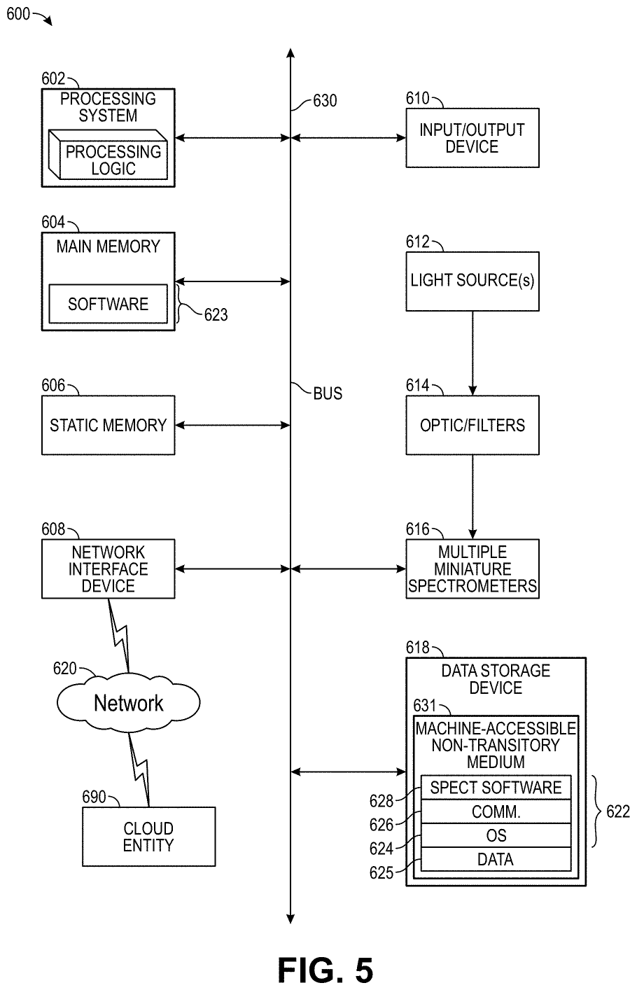

FIG. 5 illustrates a diagrammatic representation of a machine in the exemplary form of a computer system or device 600 within which a set of instructions, for causing the machine to perform any one or more of the methodologies discussed herein, may be executed, in accordance with one embodiment. In alternative embodiments, the machine may be connected (e.g., networked) to other machines in a LAN, an intranet, an extranet, or the Internet. The machine may operate in the capacity of a server or a client machine in a client-server network environment, or as a peer machine in a peer-to-peer (or distributed) network environment. The machine may be a personal computer (PC), a tablet PC, a set-top box (STB), a Personal Digital Assistant (PDA), a cellular telephone, a mobile device, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines tha individually or jointly execute a set (or multiple sets) of instructions to perform any one or more of the methodologies discussed herein.

The exemplary device 600 (e.g., multi-spectral detection device or system 600 that integrates optical components of two or more mini-spectrometers) includes a processing system 602, a main memory 604 (e.g., read-only memory (ROM), flash memory, dynamic random access memory (DRAM) such as synchronous DRAM (SDRAM) or Rambus DRAM (RDRAM), etc.), a static memory 606 (e.g., flash memory, static random access memory (SRAM), etc.), and a data storage device 618, which communicate with each other via a bus 630.

The multi-spectral detection system 600 is configured to execute instructions to perform algorithms and analysis to determine at least one of specific substances detected.

The multi-spectral detection system 600 is configured to collect data and to transmit the data directly to a remote location such as cloud entity 690 that is connected to network 620. A network interface device 608 transmits the data to the network 620. The data collected by the system 600 can be stored in data storage device 618 and also in a remote location such as cloud entity 690 for retrieval or further processing.

Processing system 602 represents one or more general-purpose processing devices such as a microprocessor, central processing unit, or the like. More particularly, the processing system 602 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing system 602 may also be one or more special-purpose processing devices such as an application specific integrated circuit (ASIC), a field programmable gate array (FPGA), a digital signal processor (DSP), network processor, or the like. The processing system 602 is configured to execute the processing logic 640 for performing the operations and steps discussed herein. The processing system 602 may include a signal processor, AI module, digitizer, int., and synch detector.

Excitation energy from one or more excitation (i.e., light) source(s) 612 is directed through a spectral filter at target material(s) in order to generate an emission. Although light source(s) 612 are shown, the disclosed embodiments may include any number of excitation sources, including using only a single light source. Preferably, light source or sources may produce narrow-band energy of about 10 nanometers or less. More preferably, the narrow-band energy is about 3 nanometers or less. Light sources may be turned on and off quickly, such as in a range of about or less than 0.01 of a second. Preferably, light sources may be turned on and off within a time period of about 0.001 second.

Emission energy from the targeted material is detected through an optic/low-pass spectral filter 614 prior to being analyzed by a spectrometer of multiple miniature spectrometers 616. Visible light filter may be located in front of optic/low-pass spectral filter 614. Visible light filter helps prevent a large spectrum of light from entering the system so that the large spectrum does not overload the subsequent components with information.

Spectrometers 616 [or array of detectors] are coupled to a synchronous detector of the processing system 602. A miniature spectrometer design platform utilizes multiple spectrometers 616 including UV Fluorescence spectrometer, UV absorption/reflection spectrometer, a near-IR (NIR) spectrometer, a Raman spectrometer, or FTIR spectrometer.

The device 600 may further include a network interface device 608. The device 600 also may include an input/output device 610 or display (e.g., a liquid crystal display (LCD), a plasma display, a cathode ray tube (CRT), or touch screen for receiving user input and displaying output.

The data storage device 618 may include a machine-accessible non-transitory medium 631 on which is stored one or more sets of instructions (e.g., software 622) embodying any one or more of the methodologies or functions described herein. The software 622 may include an operating system 624, spectrometer software 628 (e.g., multispectral detection software), and communications module 626. The software 622 may also reside, completely or at least partially, within the main memory 604 (e.g., software 623) and/or within the processing system 602 during execution thereof by the device 600, the main memory 604 and the processing system 602 also constituting machine-accessible storage media. The software 622 or 623 may further be transmitted or received over a network 620 via the network interface device 608.

The machine-accessible non-transitory medium 631 may also be used to store data 625 for measurements and analysis of the data for the detection system. Data may also be stored in other sections of device 600, such as static memory 606, or in cloud entity 690.

In one embodiment, a machine-accessible non-transitory medium contains executable computer program instructions which when executed by a handheld optical device (e.g., system 100, EPS system 300, EPS system 400) cause the system to perform any of the methods discussed herein.

The disclosed embodiments allow for an extensive number of applications including detecting and characterizing pathogens and biomarkers. A non-exclusive list of medical applications includes, but is not limited to:

measuring pathogenic viruses in bodily fluids, in particular SARS-COV-2, which can be measured in mass facilities, such as stadiums and concert halls;

rapid determination of infection;

medical diagnostic testing by detection of validating clinical recommendations for treatment, especially for diseases where onset of critical patient conditions is likely to result in rapidly declining health; and

rapid determination in a physician's office or elsewhere of the presence or absence of viral or bacterial pathogens in a patient in order to direct proper treatment).

Applications of biomarkers include measurement of biomarkers in diseases include, but not limited to:

Acute Bronchitis, Acute Respiratory Distress Syndrome (ARDS), Alpha-1 Antitrypsin Deficiency, Asbestosis, Asthma, Blood Culture, Bone Disease, Bronchiectasis, Bronchiolitis. Bronchiolitis Obliterans with Organizing Pneumonia (BOOP), Bronchopulmonary Dysplasia, Byssinosis, Cancers, Chronic Obstructive Pulmonary Disease (COPD), Chronic Thromboembolic Pulmonary Hypertension (CTEPH), Coccidioidomycosis, Cough, Cryptogenic Organizing Pneumonia (COP), Cystic Fibrosis (CF), Deep Vein Thrombosis (DVT)/Blood Clots, Emphysema, Encephalitis, Enteric pathogens, Exosomal biomarkers for cancer and other diseases, Gastrointestinal Disease, Hantavirus Pulmonary Syndrome (HPS), Histoplasmosis, Human Metapneumovirus (hMPV), Hypersensitivity Pneumonitis, Idiopathic Pulmonary Fibrosis (IPF), Influenza (Flu), Interstitial Lung Disease (ILD), Intubation infections, Kidney Disease, Liver Disease, Lung Cancer, Lymphangioleiomyomatosis (LAM), Lymphoma and Leukemia, Meningitis, Mesothelioma, Middle Eastern Respiratory Syndrome (MERS), Nontuberculosis Mycobacteria (NTM), Nosocomial Infections, Pancreatic Cancer, Pertussis, Pneumoconiosis, Pneumonia, Primary Ciliary Dyskinesia (PCD), Pulmonary Arterial Hypertension (PAH), Pulmonary Fibrosis (PF), Pulmonary Hypertension, Respiratory Infections, Respiratory Syncytial Virus (RSV), Sarcoidosis, Severe Acute Respiratory Syndrome (SARS), Shortness of Breath, Silicosis, Sleep Apnea (OSA), Sudden Infant Death Syndrome (SIDS), and Tuberculosis (TB).

Other measurement applications (including, but not limited to):

Kidney diseases, any material with biomarkers whose absorption spectra are in the MIR wavelength range, Cannabis QC/QA measurements, Oil and gas processing and contaminants, Spirits and counterfeits, Drugs and counterfeits, Illicit drugs, Industrial chemicals and constituents, Explosives, Indoor/outdoor air quality, Water quality, Effluent/sewage analysis, Agricultural and forestry, Breath analysis, Hospital air monitoring, Anesthetic Gases, In vivo imaging, and Food safety/quality/adulteration.

In one example, an integrated UV Spectrometer Platform (iUVS) was used for detection of a viral pathogen from a panel of 6 viruses. The following is a detailed description of the methodology and the results achieved.

### MATERIALS

The testing was done with a panel of the following viruses:

1. Human CoV-2 virus—1.91 mg/ml

2. Human Coronavirus OC43—0.96 mg/ml

3. Human Coronavirus NL63—1.94 mg/ml

4. Influenza Virus A [A/Wisconsin/67/2005]H3N2 virus—0.87 mg/ml

5. Influenza Virus B [B/Florida/07/2004]—1.28 mg/ml

6. Respiratory Syncytial Virus A—2.1 mg/ml

### 3 METHODS AND OBJECTIVES

A spectrofluorometer that combines, simultaneously, the functions of fluorescence and absorbance spectrometers was used. Thanks to its high-speed built-in CCD detector, the spectrofluorometer can acquire a full spectrum from 220 nm to 1,100 nm rapidly. Fluorescence excitation wavelengths from 220 nm-500nm were used for all these data, and the emission wavelength range was 250 nm-650 nm. In one example, the wavelength increment step size for the fluorescence data is 5 nm. Absorption was measured by scanning from 220-500 nm in 2 nm steps.

A purified CoV-2 virus was diluted into two different solutions. One was 0.5% Triton X-100/0.6 M KCl which is the buffer the virus was stored in after purification. The other was pooled human saliva.

Specificity: 1:5 dilutions of all the viruses listed above were made in dilution buffer and in human saliva and fluorescence and absorption were measured for all the viruses.

Sensitivity: Sensitivity measurements were made for CoV-2 virus. The virus was diluted both in buffer and saliva—1:5, 1:20 and 1:40.

The present design establishes that the multispectral Enhanced Photodetection Spectroscopy (EPS) technique, integrating two spectroscopic techniques, can detect and identify CoV-2 with high sensitivity and fidelity.

The results presented below, demonstrate unambiguously that multispectral EPS technology with data fusion and applying machine learning, can in fact detect CoV-2 in saliva in the relevant concentration range to identify an infected individual.

Objective 1—Measure Inactive Virus in Saliva with UV Fluorescence and UV Absorption Processes. Multispectral measurements were made on three separate thermally weakened coronaviruses, including SARS-CoV-2 (Covid-19), coronavirus NL63, and coronavirus OC43. In addition, measurements were made on Influenza A and B and RSV, which are non-similar viruses to the coronavirus group. The measurements on this panel of viruses provides evidence about the level of specificity that can be obtained with this multispectral approach.

Measurements of dilutions of the virus samples in buffer in the ratio from 1:1 to 1:100 were made to determine the sensitivity of measurement, one key aspect of developing a diagnostic tool.

Pure virus samples were prepared for benchtop spectrometers (UV Fluorometer, UV absorbance spectrometer), and placed in sample holders. Following spectral analysis of viruses in buffer, the same experiments were done with viruses diluted in saliva. Data fusion were used to analyze the data.

Objective 2—Calculate Sensitivity and Repeatability

Ten sets of measurements were performed under Objective 1. The data were analyzed to determine repeatability of identification of the viruses. Analysis of the dilutions prepared in Objective 1 were used to allow determination of the sensitivity of the proposed method for virus testing.

The data were analyzed with the machine learning efforts to provide further discrimination of the spectral components. With two different spectroscopic processes, machine learning for pattern recognition is expected to provide a powerful tool to differentiate between viruses and provide quantification based on amplitude input from each process.

Objective 3—Algorithm Design

Preliminary data analysis and testing of a variety of standard algorithms for spectral detection/classification and unmixing was done including standard optimization and regression algorithms and dictionary-based learning.

Concentrations measured were in the 4×10⁸ copies/ml (viral load) range and further dilutions as described above. This concentration is similar to that of a typical saliva sample of an infected person. Our data show clearly that the signal-to-noise ratio even in the raw data support accurate measurements of SARS-CoV-2 in saliva at the desired concentration of <10⁸ copies/ml (see data treatment below).

The results indicate that the measurement technique of combining UV absorption and UV fluorescence, with data fusion and machine learning, will be able to measure concentrations down to ˜10³ copies/ml (viral load) range, which is roughly in the realm of that achieved with the gold standard PCR technique.

If needed, combining the data with a third spectroscopy (UV reflectance) would improve the already impressive results obtained so far, and this addition is easily accomplished in our preliminary instrument design. This contemplated third spectroscopy addition will have no impact on cost or schedule, since the components required will already exist with the two main spectroscopies. However, the results achieved indicated that this may be superfluous.

### Data Fusion and Machine Learning Structure

During initial analysis, the following was provided: (1) software and tool development for the analysis of spectra, (2) initial data fusion and analysis and visualization, and (3) a comprehensive plan for implementation of several machine learning/AI pipelines to extract additional information from spectra subject to data fusion.

An extensive search of available, open-source software and tools for analyzing and visualizing spectroscopic data was conducted. The goal was to pick software that gives us the maximum flexibility, is modular and easy to customize in our own pipeline and had good documentation and was well-supported with little software bugs or idiosyncrasies to the code implementation. It was determined that our pipeline would consist of two main parts: (1) data pre-processing and visualization using MATLAB, and (2) feature extraction and machine learning using Python. A number of MATLAB toolboxes were investigated. This decision was made due to the relative strengths of each computing platform for the respective tasks. It was determined that IRootLab was the most promising software to perform data visualization and analysis. The ability to perform advanced visualizations such as feature histograms and biomarker plots will be useful for the data analysis of novel coronavirus in samples.

Using the prototype software methodology, we conducted preliminary data analysis of samples of inert virus in both buffer and saliva solutions. There are two main types of data being analyzed: an absorbance spectra and a fluorescence emission spectra when the sample is excited by different wavelengths (220 nm-290 nm). Several common respiratory viruses were tested including CoV-2, NL63, OC43, Influenza A, Influenza B, and RSV. We utilized MATLAB to read in the raw spectra and to plot them for visualization.

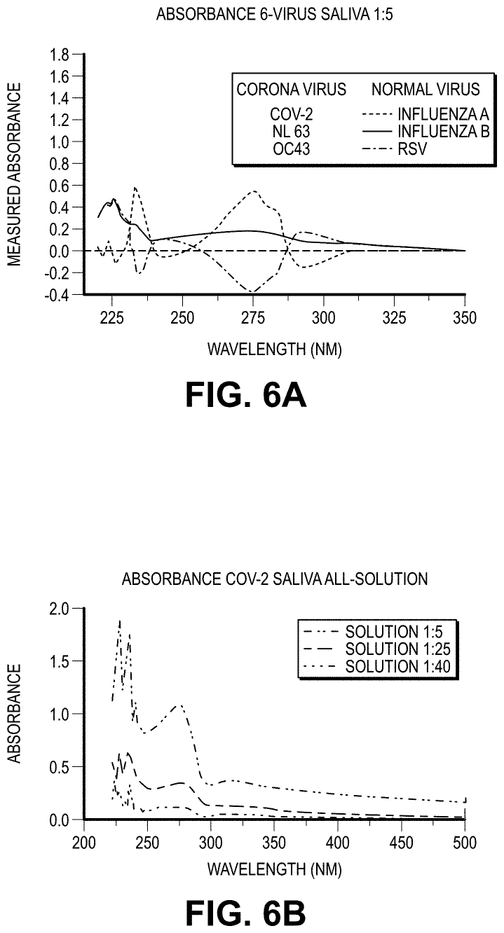

FIG. 6A shows plots of the absorbance spectra for the various viruses in saliva solutions, with a 1:5 ratio. There are different spectral shapes occurring for different viruses, but the closest to the CoV-2 is the NL63 measurement which shares several spectral features. It will be a goal of the machine learning to help disambiguate between these viruses.

Another experiment was conducted to look at the effects of solution concentration for the absorbance spectra for CoV-2. As can be seen in FIG. 6B, the amplitude (and less so the shape) of the spectra can change in absorbance significantly with respect to the ratio, with absorbance on a y-axis decreasing as the virus becomes more diluted. This could potentially help determine the concentration or strength of the viral load within a sample.

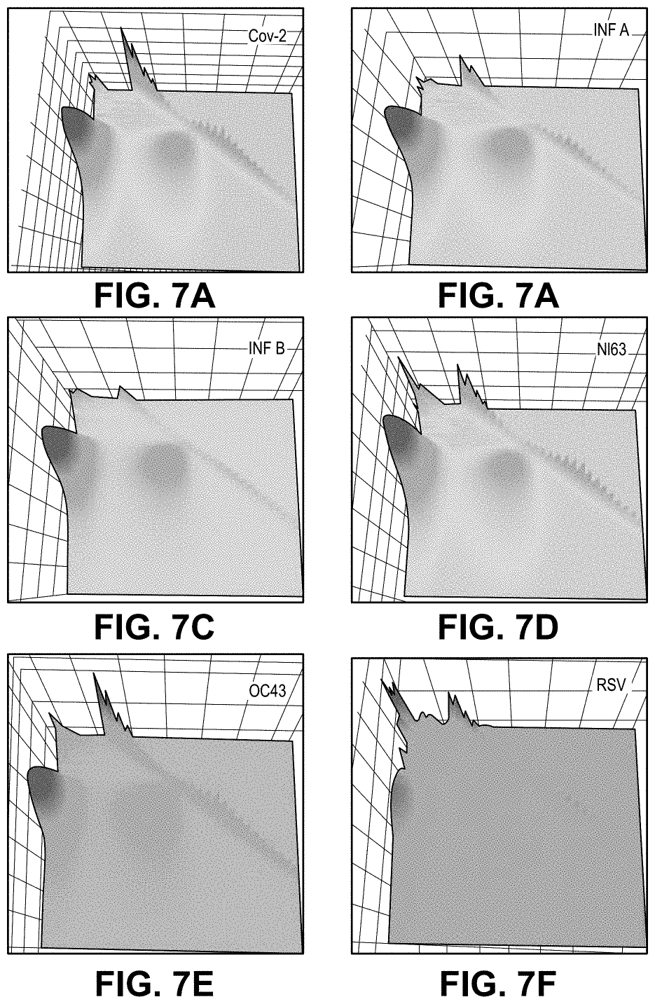

FIGS. 7A-7F illustrate fluorescence (emission-excitation) spectra for the 6 viruses (including CoV-2), where X and Y axes represent the excitation and emission wavelengths, respectively, and the Z axis is the intensity. The 3D representation visually demonstrates the difference between viruses where different excitation wavelengths result in different emission spectra. FIG. 7A illustrates a spectrum in 3D for CoV-2, FIG. 7B illustrates a spectrum in 3D for INF A, FIG. 7C illustrates a spectrum in 3D for INF B, FIG. 7D illustrates a spectrum in 3D for NL63, FIG. 7E illustrates a spectrum in 3D for OC43, and FIG. 7F illustrates a spectrum in 3D for RSV.

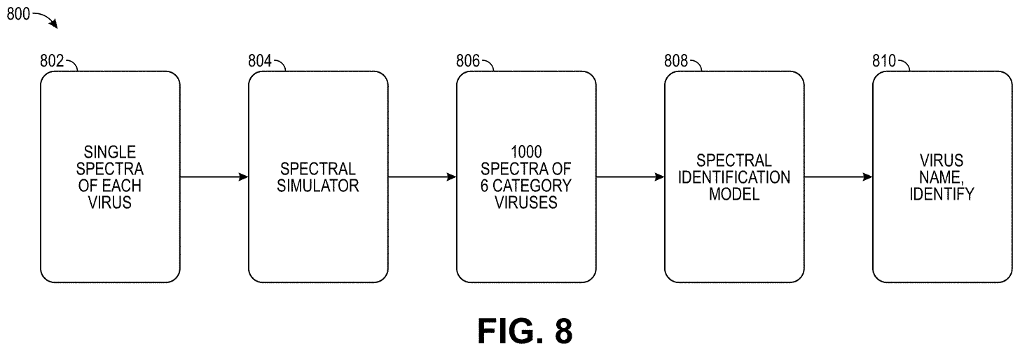

Next, preliminary machine learning feature extraction and classification was performed. Given limited data, a test was performed with the following procedure illustrated in FIG. 8. Namely, the present design takes spectra 802 from each type of virus, and simulates with a spectral simulator 804 variation in the spectra due to different types of multiplicative and additive noise. Using these generated spectra 806, the design performs feature extraction and unsupervised machine learning techniques such as principal component analysis (PCA) to build a spectral identification model 808 that determines a virus name and identity 810.

The samples used for these measurements were purified solutions. Adding artificial noise is a way to simulate real-world conditions, where the saliva may be analyzed after meals and drinks, and with possible contamination with other viruses and bacteria and fragments thereof.

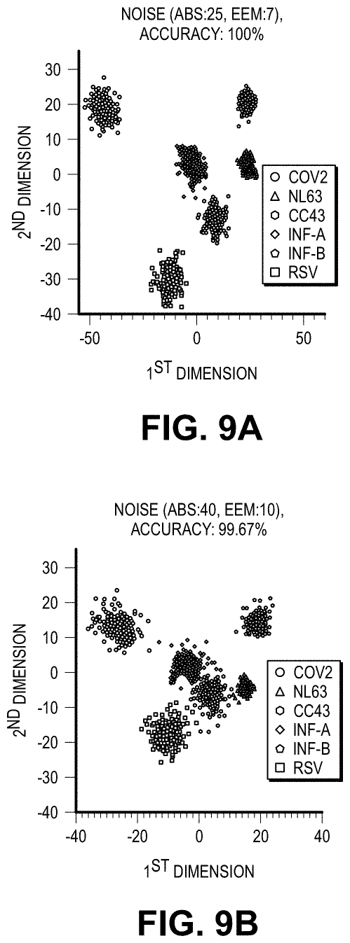

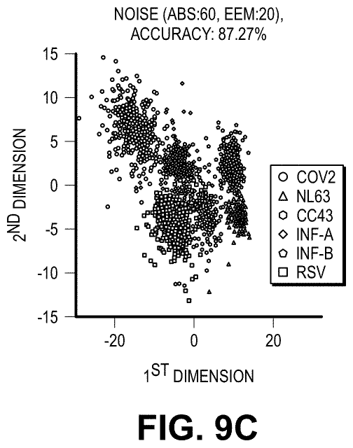

FIGS. 9A, 9B, and 9C show the results of our PCA feature extraction in terms of scatter plots visualizing the principal component analysis. As you can see, the method is able to disambiguate the viruses clearly in both absorbance and emission spectra.

To develop a classifier, the present design uses a weighted K-nearest neighbors (KNN) algorithm which allows us to predict an accuracy for virus detection as well as a confidence score for those measurements.

Next data fusion was performed between the absorbance and emission spectra as displayed in FIGS. 9A, 9B, and 9C. Data fusion is a task where information from multiple sources is combined to extend data analysis and enable new capabilities. For instance, this could improve data analysis to higher performance with respect to a given metric (e.g., accuracy, precision, confidence). Data fusion typically works well when the two data sources have complementary strengths and weaknesses for the task at hand. However, it is not straightforward to implement data fusion, and typically machine learning and artificial intelligence techniques are leveraged to find optimal ways to perform this combination.

In our case, data fusion was performed between the absorption and emission spectra collected with our spectrometers. The main goal for doing so is to improve detection and identification of viruses with higher accuracy and confidence than using only one of the two spectra modalities alone. In addition, we plan to investigate the feasibility of determining viral concentration or the percentage of the sample that contains the virus. This problem, known as spectral unmixing, seeks to separate a given sample into the percentages (or abundances) of various materials/compounds. To enable this additional functionality, we will require gathering data samples to help train machine learning/AI algorithms to perform data fusion. This will help extend the capabilities of our spectrometer and data analysis pipeline.

FIGS. 9A, 9B, and 9C plot the first two dimensions of the principal component analysis (PCA) feature that were extracted from our original viral samples (using the data augmentation with noise method described earlier). Each plot allows visualizing each generated spectra's features plotted in a color for the virus family it belongs too. For machine learning/AI features, it is desirable to have the clusters of features for each virus to be grouped together but separate in distance from other clusters to enable distinguishability for the machine learning algorithm. As can be seen, as the noise increases (25%->60% for absorbance, and 7%->20% for emission spectra), the virus clusters start to break apart and get mixed together. However, our classifier, based on weighted K Nearest Neighbors (KNN) still is highly effective with only a small drop in accuracy. This shows the benefits of machine learning in that it can make the detection and identification of these viruses' spectra under noisy conditions. Testing the robustness of our features will be evaluated with a large-scale dataset of samples collected by the spectrometer, as we train neural network and machine learning pipelines on these extracted features.

Our data fusion plan is to first extract spectral features from both the absorption and emission spectra. These features are typically represented as numerical vectors that encode salient information about each spectrum. Then these features will be jointly combined and inputted into a neural network. This neural network, called a Long Short Term Memory Network (LSTM), will utilize the two features to extract enough statistical information to make a decision of what type of virus it is. Further, our data fusion can potentially help improve auxiliary tasks such as determining the viral load concentration present in a given sample. Data fusion can be leveraged to get the most performance out of our spectrometer.

Our data analysis supports that our software pipeline could process raw data from the spectrometer and do initial analysis of the spectra. The present design also implements a preliminary feature extraction and machine learning classifier to identify the viruses.

The present design implements a full machine learning pipeline aimed at various tasks to help with spectral identification/detection.

Sample Viability and Characterization of Data Quality—The first main task is to determine if a given spectrum from a sample is viable and can be processed further for advanced diagnostics. This is an important step as our pipeline is designed to be scalable for large processing loads with numerous samples, and it is important to have rejection criteria. After a data sample is deemed viable, basic preprocessing is performed to characterize the data sample including quantifying the number of spectral channels, basic statistics of the spectrum that can be queried for analysis and determining the signal-to-noise ratio for the spectrum.

This design leverages several advanced signal processing and machine learning algorithms to develop the rejection criteria. For many data samples, this design can occasionally get distorted or errors in the spectra due to an instrument error or miscalibration. This this design will develop quick statistical rejection threshold techniques based on moving or weight averages for spectral channels, based on anomaly detection theory. The goal of these algorithms will be to parse a large corpus of spectra and determine which spectra are anomalies and have unusual structure in their spectra that could indicate an instrument or calibration error during data capture. For more advanced methods (if needed), this design will leverage Bayesian priors to test the likelihood of an instrument/calibration error.

Data Feature Extraction—One of the key steps to a machine learning pipeline is to extract meaningful data features to later perform inference and other analysis tasks. These features can either be manually designed based on domain knowledge or learned directly from training data and dataset statistics. In our pipeline, this present design investigates both strategies to determine the optimal features for our downstream applications.

Sample manual features to be used include simple statistics (mean, average, peak, standard deviation, windowed averages), power spectral density, FFT coefficients, and wavelet-based features. In addition, this design will perform principal component analysis (PCA) using singular value decomposition of data hypercubes and use the derived principal components as a natural representation for the data.

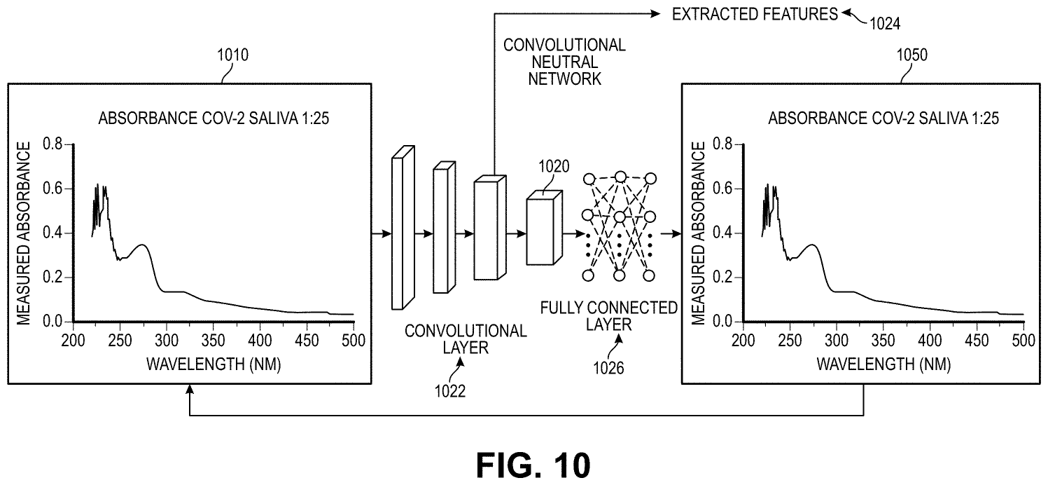

For learned features, this design plans to use two types of features: features from a self-supervised autoencoder, and features from trained supervised networks. In the former case, a Convolutional Neural Network (CNN), Long Short Term Memory Network (LSTM), and Gated Recurrent Unit (GRU) layers will be optimized to take input spectra 1010 and output the same spectra 1050, but after going through a compression/bottleneck stage in the middle of the neural network 1020 as shown in FIG. 10 below. This allows the network to learn good features to perform signal reconstruction, and which correlate well with good features for discriminative tasks such as spectral detection and identification.

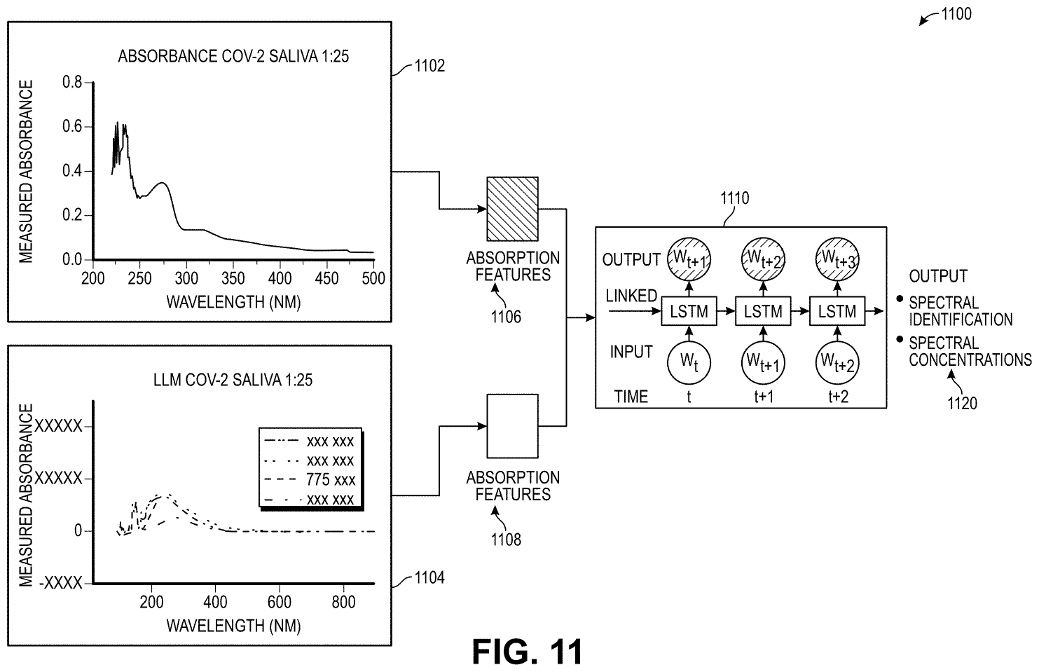

In one example, this design performs spectral detection and identification of novel coronavirus as compared to other spectra from the multi-spectral system. A data set of known coronavirus spectra, collected from various sources, will be developed. Then, given this dataset, feature extraction will be performed and a neural network built to identify coronavirus spectra from these features. As noted earlier, coronavirus spectra are identifiable from their peak at certain wavelengths, and thus simple algorithms can perform identification. However, for robust detection and identification, particularly in the case of noise, other chemicals and materials can be present in the sample including other proteins viruses, bacteria, and fragments thereof. To solve this issue, this design will distort and augment spectra to be more difficult and show that our machine learning-based methods can still overcome traditional signal processing estimation methods in these challenging scenarios. The proposed machine learning pipeline 1100 is shown in FIG. 11. The machine learning pipeline 1100 includes input spectra 1102 and 1104, absorption features 1106, emission features 1108, a CNN 1110, and output 1120.

There are several key metrics of interest in our machine learning pipeline. This includes:

Detection accuracy

Confidence of detection accuracy [p-value based on statistical tests]

Type I error [detecting coronavirus erroneously]

Type II error [failing to detect coronavirus]

Uncertainty quantification for our machine learning methods, including variability, ensemble.

The analyzed data shows conclusively that coronavirus (CoV-2) can be detected in saliva and distinguished from other viruses. Six different viruses were tested, and spectra analyzed with added noise levels to simulate the real-world condition of contaminations in individual saliva.

Machine learning based on data fusion from UV absorption and UV excitation-emission spectra unambiguously demonstrated the power of this technique to unravel the key identifying features from the noisy spectra.

This sets the stage for developing an integrated multispectral instrument with embedded machine learning trained on large data sets. The preliminary data treatment possible with the limited data sets that could be generated still clearly demonstrated that this will be an instrument with the capability to “learn” the signatures of other viruses and new pandemic viruses as they inevitably will appear.

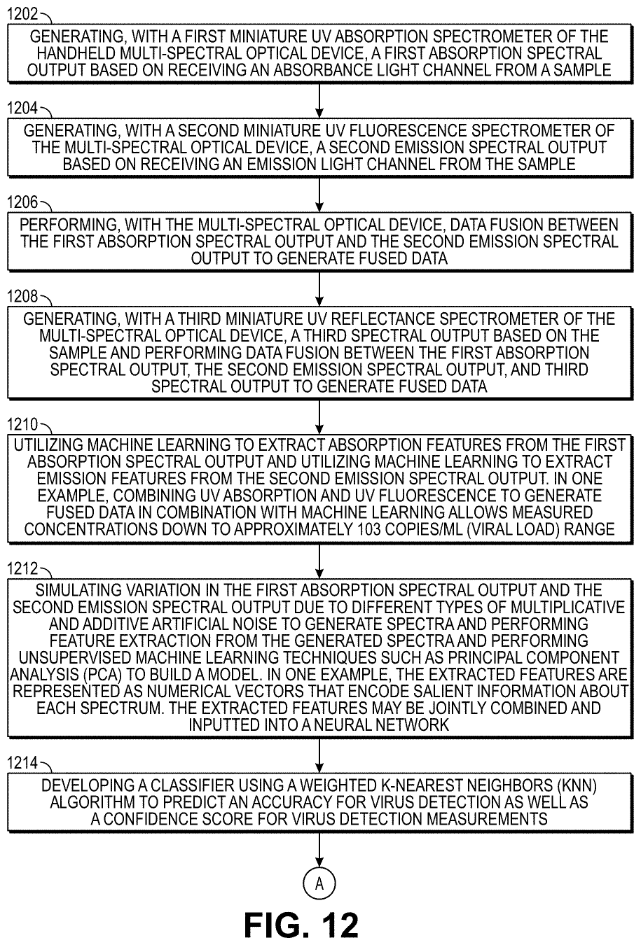

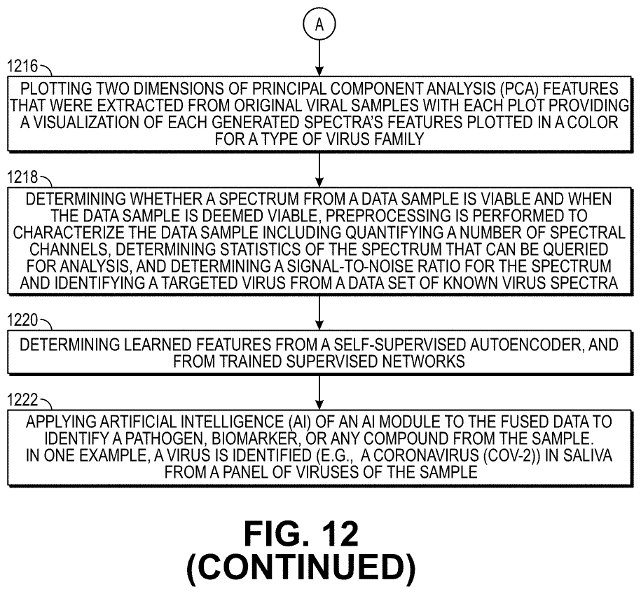

FIG. 12 illustrates a method for operations of a handheld multi-spectral optical device in accordance with one embodiment. At operation 1202, the method includes generating, with a first miniature UV absorption spectrometer of the handheld multi-spectral optical device, a first absorption spectral output based on receiving an absorbance light channel from a sample. At operation 1204, the method includes generating, with a second miniature UV fluorescence spectrometer of the multi-spectral optical device, a second emission spectral output based on receiving an emission light channel from the sample. At operation 1206, the method includes performing, with the multi-spectral optical device, data fusion between the first absorption spectral output and the second emission spectral output to generate fused data.

At optional operation 1208, the method includes generating, with a third miniature UV reflectance spectrometer of the multi-spectral optical device, a third spectral output based on the sample and performing data fusion between the first absorption spectral output, the second emission spectral output, and third spectral output to generate fused data.

At operation 1210, the method includes utilizing machine learning to extract absorption features from the first absorption spectral output and utilizing machine learning to extract emission features from the second emission spectral output. In one example, combining UV absorption and UV fluorescence to generate fused data in combination with machine learning allows measured concentrations down to approximately 10³ copies/ml (viral load) range.

At operation 1212, the method includes simulating variation in the first absorption spectral output and the second emission spectral output due to different types of multiplicative and additive artificial noise to generate spectra and performing feature extraction from the generated spectra and performing unsupervised machine learning techniques such as principal component analysis (PCA) to build a model. In one example, the extracted features are represented as numerical vectors that encode salient information about each spectrum. The extracted features may be jointly combined and inputted into a neural network.

At operation 1214, the method includes developing a classifier using a weighted K-nearest neighbors (KNN) algorithm to predict an accuracy for virus detection as well as a confidence score for virus detection measurements.

At optional operation 1216, the method includes plotting two dimensions of principal component analysis (PCA) features that were extracted from original viral samples with each plot providing a visualization of each generated spectra's features plotted in a color for a type of virus family.

At operation 1218, the method includes determining whether a spectrum from a data sample is viable and when the data sample is deemed viable, preprocessing is performed to characterize the data sample including quantifying a number of spectral channels, determining statistics of the spectrum that can be queried for analysis, and determining a signal-to-noise ratio for the spectrum and identifying a targeted virus from a data set of known virus spectra.

At operation 1220, the method includes determining learned features from a self-supervised autoencoder, and from trained supervised networks.

At operation 1222, the method includes applying artificial intelligence (AI) of an AI module to the fused data to identify a pathogen, biomarker, or any compound from the sample. In one example, a virus is identified (e.g., a coronavirus (CoV-2)) in saliva from a panel of viruses of the sample.

It will be apparent to those skilled in the art that various modifications and variations can be made in the disclosed embodiments without departing from the spirit or scope of the invention. Thus, it is intended that the present invention covers the modifications and variations of the embodiments disclosed above provided that the modifications and variations come within the scope of any claims and their equivalents.

What is claimed is:

1. A method comprising:
generating, with a first miniature UV absorption spectrometer of a multi-spectral optical device, a first absorption spectral output based on receiving an absorbance light channel from a sample;
generating, with a second miniature UV fluorescence spectrometer of the multi-spectral optical device, a second emission spectral output based on receiving an emission light channel from the sample; and
performing, with the multi-spectral optical device, data fusion between the first absorption spectral output and the second emission spectral output to generate fused data.

2. The method of claim 1, further comprising:
applying artificial intelligence (AI) of an AI module to the fused data to identify a coronavirus (CoV-2) in saliva from a panel of viruses of the sample.

3. The method of claim 1, further comprising:
utilizing machine learning to extract absorption features from the first absorption spectral output; and
utilizing machine learning to extract emission features from the second emission spectral output.

4. The method of claim 1, further comprising:
generating, with a third miniature UV reflectance spectrometer of the multi-spectral optical device, a third spectral output based on the sample; and
performing data fusion between the first absorption spectral output, the second emission spectral output, and third spectral output to generate fused data.

5. The method of claim 1, wherein combining UV absorption and UV fluorescence to generate fused data in combination with machine learning allows measured concentrations down to approximately 103 copies/ml (viral load) range.

6. The method of claim 1, further comprising:
simulating variation in the first absorption spectral output and the second emission spectral output due to different types of multiplicative and additive artificial noise to generate spectra; and
performing feature extraction from the generated spectra and performing unsupervised machine learning techniques such as principal component analysis (PCA) to build a model.

7. The method of claim 6, wherein the extracted features are represented as numerical vectors that encode salient information about each spectrum.

8. The method of claim 7, wherein the extracted features are jointly combined and inputted into a neural network.

9. The method of claim 1, further comprising:
developing a classifier using a weighted K-nearest neighbors (KNN) algorithm to predict an accuracy for virus detection as well as a confidence score for virus detection measurements.

10. The method of claim 1, wherein the multi-spectral optical device is a handheld multi-spectral optical device.

11. The method of claim 1, further comprising:
plotting two dimensions of principal component analysis (PCA) features that were extracted from original viral samples with each plot providing a visualization of each generated spectra's features plotted in a color for a type of virus family.

12. The method of claim 1, further comprising:
determining whether a spectrum from a data sample is viable; and
when the data sample is deemed viable, preprocessing is performed to characterize the data sample including quantifying a number of spectral channels, determining statistics of the spectrum that can be queried for analysis, and determining a signal-to-noise ratio for the spectrum; and
identifying a targeted virus from a data set of known virus spectra.

13. The method of claim 1, further comprising:
determining learned features from a self-supervised autoencoder, and from trained supervised networks.

14. A machine-accessible non-transitory medium contains executable computer program instructions which when executed by a handheld optical device causes the handheld optical device to perform a method comprising:
obtaining a first absorption spectral output from a first miniature UV absorption spectrometer of the handheld optical device;
obtaining a second emission spectral output from a second miniature UV fluorescence spectrometer of the handheld optical device;
performing data fusion between the first absorption spectral output and the second emission spectral output to generate fused data.

15. The machine-accessible non-transitory medium of claim 14, the method further comprising:
applying artificial intelligence (AI) of an AI module to the fused data to identify a coronavirus (CoV-2) in saliva from a panel of viruses of the sample.

16. The machine-accessible non-transitory medium of claim 14, the method further comprising:
utilizing machine learning to extract absorption features from the first absorption spectral output; and
utilizing machine learning to extract emission features from the second emission spectral output.

17. The machine-accessible non-transitory medium of claim 14, further comprising:
generating, with a third miniature UV reflectance spectrometer, a third spectral output based on the sample; and
performing data fusion between the first absorption spectral output, the second emission spectral output, and third spectral output to generate fused data.

18. The machine-accessible non-transitory medium of claim 14, wherein combining UV absorption and UV fluorescence to generate fused data in combination with machine learning allows measured concentrations down to approximately 103 copies/ml (viral load) range.

19. The machine-accessible non-transitory medium of claim 14, further comprising:
simulating variation in the first absorption spectral output and the second emission spectral output due to different types of multiplicative and additive artificial noise to generate spectra; and
performing feature extraction from the generated spectra and performing unsupervised machine learning techniques such as principal component analysis (PCA) to build a model.

20. The machine-accessible non-transitory medium of claim 19, wherein the extracted features are represented as numerical vectors that encode salient information about each spectrum.
